(19) United States
(12) Patent Application Publication
(10) Pub. No.: US 2023/0263938 A1
(43) Pub. Date: Aug. 24, 2023

(54) Dental Coating Composition and Method of Use

(71) Applicant: Mycone Dental Supply Co., Inc., Gibbstown, NJ (US)

(72) Inventors: Kevin SHERAN, Haddonfield, NJ (US); William GROSS, Moorestown, NJ (US)

(21) Appl. No.: 18/113,560

(22) Filed: Feb. 23, 2023

Related U.S. Application Data
(60) Provisional application No. 63/313,264, filed on Feb. 23, 2022.

(51) Int. Cl. A61L 27/34; A61C 13/08; A61K 6/20; A61K 6/62; B33Y 80/00

(52) U.S. Cl. CPC A61L 27/34; A61C 13/082; A61K 6/20; A61K 6/62; B33Y 80/00; A61L 2430/12; A61L 2300/802; A61L 2420/02; A61L 2420/06; A61L 2420/04

(57) ABSTRACT

The dental coating composition of the present invention is suitable to coat dental prosthetics. The composition is a mixture of a (meth)acrylate dimer, a (meth)acrylate crosslinker, a photoinitiator, and a silicone surfactant, and optionally, a (meth)acrylate monomer with a heteroalkyl group, a colorant, special effects pigment, particles, and other additives. Incorporating particles in the dental coating formulation provides a “character” to the surface of the dental prosthetic, increasing the esthetic appeal of the prosthetic. The dental coating thus may make the parts of the dental prosthetic appear to approximate gingivae, or gum. The dental coating composition may be applied to a surface of an unfinished dental prosthesis prior to curing, to a partially cured prosthesis, or fully cured prosthesis.

### CROSS-REFERENCE TO RELATED APPLICATIONS

This application is related to and claims priority to U.S. Provisional Patent Application No. 63/313,264, filed on 23 Feb. 2023, and entitled “Dental Coating Composition and Method of Use” which is incorporated by reference herein in its entirety.

### FIELD OF THE INVENTION

The present invention is directed to a formulation and a method of forming a dental coating. More particularly, the invention relates to the field of actinic light-curable dental coating suitable for use with dental prosthesis.

### DESCRIPTION OF RELATED TECHNOLOGY

Interpenetrating polymer network compositions are taught in U.S. Pat. No. 4,551,486. That disclosure provides hardenable compositions useful as construction media for a wide range of applications. Particular utility is found in the dental and medical arts where such compositions are highly suitable for the formation and construction of denture base, denture baseplates, denture liners, denture repair, custom trays, veneering for crowns and bridgework, artificial teeth, veneers and repair for natural teeth, and tooth restorative fillings.

Three-dimensional fabricating material systems and methods for producing layered dental products is taught in US Patent Publication No. 2017/0360534. That publication described the designs and builds multiple layers (two layers or more) of various dental devices, specifically denture base or denture, where printed multiple layered denture base with teeth cavities to receive artificial denture tooth materials to form final dental devices, such as partial and full dentures. It can also print denture teeth. This invention also designs and prints multiple layers (two layers or more) of denture base with artificial denture teeth to form final dental devices, such as partial and full dentures. A method for manufacturing a layered denture was provided. The disclosure provided a multiple layered denture base materials for printing a denture base. The disclosure also provided a multiple layered denture tooth materials for printing artificial denture teeth. Highly shape adjustable or shape memory polymer layer(s) may be used in these multiple layered denture base forms. Different layer of material has different mechanical and physical properties to meet different need, which provide added benefits to the patients, dental professional and dental laboratory.

Coating composition for denture base, denture base with coating film, bed denture, and method for producing denture base with coating film was taught in International Patent Publication No. WO2018/143051. A coating composition for a denture base capable of forming a coating film having excellent scratch resistance on a denture base and excellent bacterial adhesion inhibition, a denture base with a coating film, a bed denture, and a denture base with a coating film was disclosed. A method of manufacturing the same was provided.

Photopolymerizable dental coating composition is disclosed in US Patent Publication No. 2003/0060534. The publication provided a photopolymerizable dental coating composition that, when applied to the surface of a dental resin material such as crown restoratives, dental fillers, and denture or denture base resins, was thoroughly cured upon irradiation with a visible light and produces a smooth, colorless and transparent cured surface after curing so that a film having superior durability, abrasion resistance and resistance to discoloration can be formed, the photopolymerizable dental coating composition includes a volatile (meth)acrylate compound, a polyfunctional acrylate as a cross-linking agent, a fluoroalkyl group constituted of at least one fluorocarbon-containing (meth)acrylate compound for improving the durability, abrasion resistance and resistance to discoloration after curing, and an acyl phosphine oxide-based polymerization initiator having a high polymerization promoting effect for monomers, which does not make the polymerized composition remain yellowish.

Surface strengthening and smoothing coat formation denture, denture surface strengthening and smoothing coating agent and method for strengthening and smoothing surface of denture was disclosed in Japanese Patent Publication JP2007244739. The problem to be solved was identified to be to provide a surface strengthening and smoothing coat formation denture capable of preventing stain and the growth of bacteria for a long period of time, a coating agent for strengthening and smoothing the surface of the denture for manufacturing the surface strengthening and smoothing coat formation denture, and a method for strengthening and smoothing the surface of the denture. The solution was disclosed to be the coating agent for strengthening and smoothing the surface of the denture is applied on the surface of the denture, which contains a silane coupling agent and a straight silicone resin being a three-dimensional oligomer with high branching extent, where a main chain is Si—O—Si, and leaving unreacted hydroxyl group. Then the surface of the denture is coated by the straight silicone resin strengthening and smoothing coat.

After hardening, breakage-resistant prosthesis base material, obtained from autopolymerizing or cold-curing compositions was taught in German patent DE102011106816. Fracture-resistant prosthesis material, obtained from an autopolymerizing or cold-polymerizing composition, which (A) contains a liquid monomer component, (B) contains a powdery component comprising one or more bead polymers, (C) at least one initiator or an initiator system for auto- or cold polymerization that at least one aliphatic urethane diacrylate resin, which is not UDMA, is present in components (A), the prosthesis material having a fracture toughness of >1.8 MPa·m\hat{ }½ and a fracture work of >400 J/m² and, after 8 weeks of storage in fully demineralized water at 50° C., the prosthesis material had a difference in yellowness index, the b-value, of at most one unit.

Coating composition and a method for producing a synthetic resin molded product having an abrasion resistant surface was taught in U.S. Pat. No. 4,199,421. That invention related to a coating composition which comprises a polyfunctional monomer having at least three acryloyloxy groups and/or methacryloyloxy groups in one molecule, a monomer having not more than two acryloyloxy group and/or methacryloyloxy groups in one molecule and optionally a photosensitizer and which can form a crosslink-hardened film excellent in abrasion resistance upon curing by irradiation with actinic radiation in air and a method for producing a synthetic resin molded product having an abrasion resistant surface

A method of treating dentures is taught in International Patent Publication No. 98/31298. That disclosure related to a method for treating a denture, comprising passing the denture through the air interface of an aqueous composition comprising a polymeric coating agent, especially a silicone polymer, having a weight average molecular weight of 1,000 or greater. That disclosure further relates to the use of a denture bath in the method. Passing the denture through the interface in this way enhances the coating of the denture with the polymer, thereby delivering improved plaque prevention

Effect of experimental photopolymerized coatings on the hydrophobicity of a denture base acrylic resin and on Candida albicans adhesion is taught in Arch. Oral Biol. vol. 58, pp 1-9, (2013).

Multilayer acrylic resin film is taught in US Patent Publication No. 2008/0090055. A multilayer acrylic resin film having two surface layers made of an acrylic resin composition consisting of from 20 to 100% by weight of a methacrylic resin and from 0 to 80% by weight of acrylic rubber particles which contains a UV absorber in an amount of 0.2 part by weight or less per 100 parts by weight of the acrylic resin composition, and at least one intermediate layer made of an acrylic resin composition consisting of from 20 to 100% by weight of a methacrylic resin and from 0 to 80% by weight of acrylic rubber particles which contains a UV absorber in an amount of at least 0.3 part by weight per 100 parts by weight of the acrylic resin composition.

Non-volatile dental compositions containing multifunctional acrylate compounds and lacking an oxygen-inhibited layer is disclosed in U.S. Pat. No. 7,081,485. Dental composite formulations containing an initiator and a multiacrylate compound was discribed. The formulations lack methyl methacrylate, a volatile, irritating, and potentially hazardous material commonly found in dental formulations. The multiacrylate compound has at least three acrylate functionalities per molecule. The formulations cure to form a surface lacking an oxygen inhibition layer. The formulations can be used as dental sealants, dental coatings, and in fingernail/toenail repair applications.

Radiation-curable compositions and cured articles was taught in U.S. Pat. No. 6,660,374. A radiation-curable composition in a liquid or solid form comprises at least one solid, non-crystalline radiation-transmissible material, dispersed in at least one cationic-curable or free-radical curable composition or mixture thereof. The solid, non-crystalline radiation-transmissible materials comprise glasses and other suitable materials that transmit (i.e., are transparent to) at least about 40% of radiation having a wavelength from about 180 to about 600 nanometers. The cationic-curable compositions comprise at least one cycloaliphatic epoxide, at least one polyol, and at least one cation-generating photoinitiator. The free-radical curable compositions comprise at least one ethylenically unsaturated compound and at least one free-radical-generating photoinitiator unless electron beam curing is used, in which case the amount of photoinitiator can be reduced or even eliminated. The solid forms of the radiation-curable compositions of the invention are useful as powder coatings for coating decorative and functional objects and that would be cured by a thermal heating flow process followed by radiation exposure. The cured compositions of the invention are useful as coatings and inks for metal, paper, plastics, glass, ceramics, and wood, as adhesives, as sealants, and as composite materials and other articles. The cured compositions of this invention also are useful in biomedical and dental applications, including prosthetic devices such as dentures; coatings, fillings, and caps for teeth; and the like.

### SUMMARY OF THE INVENTION

The present invention relates to a dental coating composition. Such a dental coating composition is used to coat dental prosthetics. The dental coating composition is a mixture of well-defined (meth)acrylates with selected additives.

Under one embodiment, the dental coating composition comprises a (meth)acrylate monomer, a (meth)acrylate dimer, a (meth)acrylate crosslinker, a photoinitiator, and a silicone surfactant. Such a dental coating composition may optionally further comprise a colorant, a brightener, or particles.

Under one embodiment, the coating composition comprises (a) about 5 wt % to about 20 wt % of a monomer selected from the group consisting of alkyl (meth)acrylate and heteroalkyl (meth)acrylate; (b) about 30 w % to about 65 wt % of a dimer selected from the group consisting of polyalkylene glycol diacrylate, aliphatic urethane di(meth)acrylate, and alkoxylated bisphenol (meth)acrylate; (c) about 25 wt % to about 40 wt % of (meth)acrylate crosslinker selected from the group consisting of aliphatic urethane poly(meth)acrylate and ethoxylated (meth)acrylate, wherein the (meth)acrylate crosslinker has a functionality of greater than 2.5; (d) a photoinitiator; and (e) a silicone surfactant.

Under another embodiment, the dental coating composition is prepared without the (meth)acrylate monomer. Under such embodiment, the dental coating composition comprises a (meth)acrylate dimer, a (meth)acrylate crosslinker, a photoinitiator, and a silicone surfactant. Such a dental coating composition may optionally further comprise a colorant, a brightener, or particles.

The monomer as used in selected embodiments of the present invention is a (meth)acrylic monomer, such as an acrylic monomer or a methacrylic monomer, or a mixture thereof. Under one embodiment, the monomer of the present invention is selected from the group consisting of alkyl acrylate, alkyl methacrylate, heteroalkyl acrylate, and heteroalkyl methacrylate. The definition of alkyl (meth)acrylate also includes cycloalkyl (meth)acrylate.

Under one embodiment of the present invention, the (meth)acrylate monomer comprises a heteroalkyl group, wherein the heteroalkyl group comprises a heteroatom that is oxygen. The heteroalkyl group may be an ether group, an ketyl group, an ester group, or other oxygen containing chemical groups.

The di(meth)acrylate as used in selected embodiments of the present invention is a diacrylate, a dimethacrylate, or a mixture thereof.

Under one embodiment, the di(meth)acrylate of the present invention is selected from the group consisting of polyalkylene glycol diacrylate, aliphatic urethane diacrylate, aliphatic urethane dimethacrylate, alkoxylated bisphenol diacrylate, and alkoxylated bisphenol dimethacrylate.

The dental coating composition also comprises a crosslinker. Under one embodiment, the crosslinker is aliphatic urethane poly(meth)acrylate or ethoxylated (meth)acrylate. Generally, a crosslinker is a multifunctional (meth)acrylate.

The photopolymerizable composition of the present invention for forming a dental coating for dental prosthetics also optionally comprises one or more photoinitiators. The photoinitiator is selected so that it is activated by photons of the wavelength associated with actinic light of a UV lamp, an LED lamp, or another lamp.

The photopolymerizable composition of the present invention under one embodiment further comprises a small amount of a colorant or special effects pigment or a combination thereof.

The photopolymerizable composition of the present application may further comprise additional ingredients, including colorants, dyes, whiteners, perfumes, thixotropes, stabilizers, anti-oxidants, and like.

The dental coating composition of the present invention for forming a dental coating for dental prosthetics also comprises one or more silicone surfactants.

Under one embodiment, the dental coating comprises particles. These particles are selected from the group consisting of fibers, flakes, sheets, crystals and rods.

One purpose of incorporating particles in the dental coating formulation is to provide a “character” to the surface of the dental prosthetic. One purpose of incorporating particles in the dental coating formulation is to provide an esthetic appeal of the dental prosthetic. The dental coating thus may make the parts of the dental prosthetic appear to approximate gingivae, or gum.

Such particles may be selected to approximate blood capillaries, or to model blood capillaries. Under one embodiment, the particle is a fine fiber. Under one embodiment, a fiber is 1 to 5 mm long, or 1.5 to 3.0 mm long. The fibers are 5 μm to 20 μm in thickness, or 10 μm to 15 μm in thickness.

The present invention is also directed to a dental prosthesis which comprises the dental coating.

Under one embodiment, the dental prosthesis is a removable dental prosthesis. Under one embodiment, the dental prosthesis is a fixed dental prosthesis.

A dental prosthesis may be either be fixed permanently or it may be removable. Permanently fixed dental prostheses use dental adhesive or screws, to attach to teeth or to dental implants.

The dental prosthesis comprising the dental coating is a denture, a partial denture, a palatal obturator, an orthodontic appliance, a dental implant, a crown, or a bridge. Under one embodiment, the dental prosthesis is a denture, a partial denture, a palatal obturator, or a dental implant.

The present invention is also directed to a method of forming a dental prosthesis.

One advantage of the present invention over the prior art is the speed and ease of which the unfinished dental prosthesis is finished. To finish manufacturing of the dental prosthesis, whether 3D printed or manufactured in another way, such a prosthesis needed to be polished to have a glossy surface appearance and optionally “characterized” with acrylic stains to give the prosthesis more a more esthetically pleasing look. To do the finishing work, the manual labor time was about 5 to 15 minutes per arch, plus the finishing materials that are consumed.

Under the Coated Acrylic Denture Method, the method includes at least three steps: (a) obtaining an unfinished dental prosthesis, wherein the unfinished dental prosthesis comprises a surface of the unfinished dental prosthesis; (b) applying the dental coating composition onto a part of the surface; and (c) curing the coating.

The unfinished dental prosthesis may be obtained by any suitable method. Such a method includes currently known methods, or methods that may be developed in the future that produce unfinished dental prosthesis.

Examples of methods used to obtain the unfinished dental prosthesis include fabrication by additive manufacturing, 3D printing, subtractive manufacturing, milling, self-cured fabrication process, heat-cured fabrication process, or injection molding.

Under one embodiment, the part of the surface to which dental coating composition is applied to is the portion of the surface that mimics the gingivae.

After the application of the dental coating composition to a part of the surface of the dental prosthesis, the dental coating composition is cured. The curing process may be done by any of suitable processes, including exposure to heat, exposure to sunlight, exposure to UV light lamps, exposure to LED light lamps, exposure to actinic light.

The present invention is also directed to a Coated/Post-Cured method of forming a dental prosthesis that includes at least the following four steps: (a) obtaining a partially-cured dental prosthesis, wherein the partially-cured denture comprises a surface; (b) curing the partially-cured dental prosthesis; (c) applying the dental coating composition onto a part of the surface; and (d) curing the dental coating composition.

One advantage of using this method is that the curing step (b) and (d) may be performed by different techniques under different conditions.

The present invention is also directed to a Green State Coated method of forming a dental prosthesis that includes at least the following three steps: (a) obtaining a partially-cured dental prosthesis, wherein the partially-cured denture comprises a surface; (b) applying the dental coating composition of any of the claims 1 to 200 onto a part of the surface; and (c) curing the partially-cured denture and the dental coating composition.

One advantage of using this method is that the curing step (c) cures both the partially-cured denture and the dental coating composition simultaneously. This combination lowers the time to cure the partially-cured dental prosthesis. Further, this combination has been found to provide superior adhesion properties.

### DETAILED DESCRIPTION OF THE INVENTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

As used herein and in the appended claims, the singular forms “a”, “an”, and “the” include plural references unless the context clearly dictates otherwise. The singular form of any class of the ingredients refers not only to one chemical species within that class, but also to a mixture of those chemical species; for example, the term “photoinitiator” in the singular form, may refer to a mixture of compounds each of which is also a photoinitiator. The term “denture” also includes the more common form “dentures”. The terms “a” (or “an”), “one or more” and “at least one” may be used interchangeably herein. The terms “comprising”, “including”, and “having” may be used interchangeably.

The abbreviations and symbols as used herein, unless indicated otherwise, take their ordinary meaning. The term “wt %” means percent by weight with respect to the entire formulation. The abbreviation “UV” stands for ultraviolet. Symbols “μm” and “mm” refer to micrometers and millimeters, respectively.

The term “about” when referring to a number means±5%. For example, the phrase “about 30 wt %” refers to a number between and including 28.500 and 31.500.

The phrase “dental coating composition” refers to a lacquer, a gel, or any other fluid, that is suitable to be applied to dental prosthetic to decorate or protect the dental prosthetic, that when hardened forms a dental coating.

The phrase “dental coating,” refers to the hardened, fully cured substance covering a part or all of the dental prosthesis. The phrase “dental coating” refers to finish product (which may then be buffed or filed), and may be composed of a single layer or multiple layers. The phrase “dental coating” is interpreted broadly, and it includes any hardened substance on a dental prosthesis due to the application of the corresponding lacquer, gel, or any other fluid.

The term “layer” refers to a single application of a dental coating composition onto a dental prosthesis. As the context dictates, the term “layer” refers to an uncured layer, or a wet layer, of the dental coating composition, or the term refers to a dried layer, or partially dried or partially cured layer of the dental coating composition.

The term “patient” refers to a person who is treated.

The term “technician” is a worker skilled or licensed in the art of manufacturing dental prosthetics.

When referring to a composition, the definition of the term “acrylate”, as referred to in the monomeric or a dimeric form, includes an ester, a salt, or a conjugate base of acrylic acid, with the formula CH₂═CH—COO—. The definition of the term “acrylate” referred to in the polymeric or oligomeric form includes the repeating unit of an ester, a salt, or a conjugate base of acrylic acid, with the formula —[CH₂—CH(COO—)]—.

The definition of the term “methacrylate” as referred to in the monomeric or dimeric form includes an ester, a salt, or a conjugate base of methacrylic acid, with the formula CH₂═C(CH₃)—COO—. The definition of the term “methacrylate” as referred to in the polymeric or oligomeric form includes an ester, a salt, or a conjugate base of methacrylic acid, with the formula —[CH₂═C(CH₃)—COO—]—.

The term “(meth)acrylate” means acrylate, methacrylate, or a mixture thereof. When referring to a compound, “(meth)acrylate” means an ester, a salt, or a conjugate base of an acrylic acid, with the formula CH₂═C(R)—COO—, wherein R is H, Me, or a mixture thereof. The definition of the term “(meth)acrylate” as referred to in the polymeric or oligomeric form includes an ester, a salt, or a conjugate base of methacrylic acid, with the formula —[CH₂═C(R)—COO—]—, wherein R is H, Me, or a mixture thereof. By extension, a monomer, oligomer, or polymer name containing as a part of its term the string “(meth)acrylate” should be interpreted as referring to the same monomer, oligomer, or polymer, that is an acrylate, methacrylate, or a mixture thereof. For example, the term “alkyl (meth)acrylate)” means “any of alkyl acrylate, alkyl methacrylate, and a mixture of alkyl acrylate and alkyl methacrylate”.

The phrase “alkyl group” relates to a linear or branched saturated hydrocarbon group, which is bound to the rest of the molecule by means of a single bond. The alkyl group may contain any number of carbons that would be appropriate for use in dental coating composition. The term “alkyl group”, unless specifically referred to otherwise, may be a branched alkyl group, a linear alkyl group. The adjective form “alkyl” without a noun that it modifies following it means an alkyl group; likewise, the term “methyl” without a noun that it modifies following it means a methyl group; etc.

Under one embodiment, the present invention relates to a dental coating composition. Such a dental coating composition is used to coat dental prosthetics.

Under one embodiment, the dental coating composition of the present invention is a fully formulated composition, meaning that dental coating composition is designed to be applied to the dental prosthetic without any further addition of ingredients, diluents, excipients, or active ingredients. Under an alternative embodiment, the dental coating composition of the present invention is only a partially formulated composition, meaning that dental coating composition may be further formulated at any time prior to the application of the dental coating to the dental prosthetic.

The dental coating composition is a mixture of well-defined (meth)acrylates with selected additives.

Under one embodiment, the dental coating composition comprises a (meth)acrylate monomer, a (meth)acrylate dimer, a (meth)acrylate crosslinker, a photoinitiator, and a silicone surfactant. Such a dental coating composition may optionally further comprise a colorant, a brightener, or particles.

The ratios of the monomer, dimer, and polyfunctional (meth)acrylate have an effect on the physical and chemical properties of the dental coating composition. Under one embodiment, the coating composition comprises (a) about 5 wt % to about 20 wt % of a monomer selected from the group consisting of alkyl (meth)acrylate and heteroalkyl (meth)acrylate; (b) about 30 w % to about 65 wt % of a dimer selected from the group consisting of polyalkylene glycol diacrylate, aliphatic urethane di(meth)acrylate, and alkoxylated bisphenol (meth)acrylate; (c) about 25 wt % to about 40 wt % of (meth)acrylate crosslinker selected from the group consisting of aliphatic urethane poly(meth)acrylate and ethoxylated (meth)acrylate, wherein the (meth)acrylate crosslinker has a functionality of greater than 2.5; (d) a photoinitiator; and (e) a silicone surfactant.

Under another embodiment, the dental coating composition is prepared without the (meth)acrylate monomer. Under such embodiment, the dental coating composition comprises a (meth)acrylate dimer, a (meth)acrylate crosslinker, a photoinitiator, and a silicone surfactant. Such a dental coating composition may optionally further comprise a colorant, a brightener, or particles.

The ratios of the dimer and the (meth)acrylate crosslinker affect the physical and chemical properties of the dental coating composition. Under one embodiment, the coating composition comprises (a) about 52 w % to about 75 wt % of a dimer selected from the group consisting of polyalkylene glycol diacrylate, aliphatic urethane di(meth)acrylate, and alkoxylated bisphenol (meth)acrylate; (c) about 20 wt % to about 43 wt % of (meth)acrylate crosslinker selected from the group consisting of aliphatic urethane poly(meth)acrylate and ethoxylated (meth)acrylate, wherein the (meth)acrylate crosslinker has a functionality of greater than 2.5; (d) a photoinitiator; and (e) a silicone surfactant.

The monomer as used in selected embodiments of the present invention is a (meth)acrylic monomer, such as an acrylic monomer or a methacrylic monomer or a mixture thereof.

An acrylic monomer is a compound containing the acrylate group of formula CH₂═CH—CO—O—. A methacrylic monomer is a compound containing the methacrylate group of formula CH₂═CMe—CO—O—.

The monomer of the present invention is selected from the group consisting of alkyl acrylate, alkyl methacrylate, heteroalkyl acrylate, and heteroalkyl methacrylate. The definition of alkyl (meth)acrylate also includes the cycloalkyl (meth)acrylate.

The (meth)acrylate monomer used in the present invention may be any acrylate monomer or methacrylate monomer that is used in dental formulations in which the curing is performed by UV light. The (meth)acrylate monomer has a formula CH₂═C(R)—COOR′, wherein R is H, Me, or a mixture thereof, and R′ is an organic group. Examples of organic group R′ include hydrocarbons, that is, aliphatic (e.g., alkyl or alkenyl), alicyclic (e.g., cycloalkyl, cycloalkenyl) compounds, and aromatic-, aliphatic-, and alicyclic-substituted aromatic compounds, as well as cyclic compounds wherein the ring is completed through another portion of the molecule (e.g., two substituents together form an alicyclic compound); groups that include hetero atoms, that is, groups that contain other than carbon in a ring or chain otherwise composed of carbon atoms, such as oxygen, nitrogen, such as groups containing non-hydrocarbon groups, such as alkoxy, amino, amido, and similar groups.

Examples of (meth)acrylate include methyl acrylate, methyl methacrylate, ethyl acrylate, ethyl methacrylate, hydroxypropyl acrylate, hydroxypropyl methacrylate, butylacrylate, butyl methacrylate, hydroxyethyl acrylate, propyl methacrylate, isobutyl methacrylate, sec-butyl methacrylate, hydroxyethyl methacrylate, butoxyethyl acrylate, butoxyethyl methacrylate, diethylaminoethyl acrylate, diethylaminoethyl methacrylate, 2-ethylhexyl acrylate, 2-ethylhexyl methacrylate, ethoxyethyl acrylate, ethoxyethyl methacrylate, t-butyl aminoethyl acrylate, t-butyl aminoethyl methacrylate, methoxyethylene glycolacrylate, methoxyethylene glycol methacrylate, phosphoethyl acrylate, phosphoethyl methacrylate, methoxy propyl acrylate, methoxy propyl methacrylate, phenoxyethylene glycol acrylate, tetrahydrofurfuryl methacrylate, phenoxyethylene glycol methacrylate, caprolactone methacrylate, methacroyloxyethyl maleate, 2-hydroxyethyl methacrylate/succinate, phthalic acid monoethyl methacrylate, phenoxypolyethylene glycol acrylate, phenoxypolyethylene glycol methacrylate, 2-hydroxy-3-phenoxypropyl acrylate, 2-hydroxy-3-phenoxypropyl methacrylate, isobornyl acrylate, isobornyl methacrylate, 3-chloro-2-hydroxypropyl acrylate, 3-chloro-2-hydroxypropyl methacrylate, and mixtures thereof.

Under one embodiment, examples of (meth)acrylate monomers comprise a (meth)acrylate selected from the group consisting of hydroxypropyl (meth)acrylate, hydroxyethyl (meth)acrylate, ethyl (meth)acrylate, propyl (meth)acrylate, butyl (meth)acrylate, isobutyl (meth)acrylate, sec-butyl (meth)acrylate, tetrahydrofurfuryl (meth)acrylate, caprolactone (meth)acrylate, (meth)acroyloxyethyl maleate, 2-hydroxyethyl (meth)acrylate/succinate, phthalic acid monoethyl (meth)acrylate, isobornyl (meth)acrylate, phenyl (meth)acrylate, benzyl (meth)acrylate, or a mixture thereof.

Under one embodiment of the present invention, the (meth)acrylate monomer comprises a hydroxyl-containing (meth)acrylate monomer.

Under one embodiment of the present invention, the (meth)acrylate monomer comprises a heteroalkyl group, wherein the heteroalkyl group comprises a heteroatom that is oxygen. The heteroalkyl group may be an ether group, an ketyl group, an ester group, or other oxygen containing chemical groups.

The alkyl group in the alkyl acrylate or alkyl methacrylate, under one embodiment, is comprises one to twelve carbon atoms. Examples C₁₋₁₂alkyl (meth)acrylate include methyl (meth)acrylate, ethyl (meth)acrylate), propyl (meth)acrylate, n-propyl (meth)acrylate, isopropyl (meth)acrylate, butyl (meth)acrylate, n-butyl (meth)acrylate, isobutyl (meth)acrylate, sec-butyl (meth)acrylate, pentyl (meth)acrylate, hexyl (meth)acrylate, heptyl (meth)acrylate, octyl (meth)acrylate, nonyl (meth)acrylate, decyl (meth)acrylate, undecyl (meth)acrylate, and dodecyl (meth)acrylate. In these examples, the alkyl groups hexyl, heptyl, octyl, nonyl, decyl, undecyl, and dodecyl may be straight chain groups or branched groups.

Examples of a branched alkyl group include: 1-methylpropyl; sec-butyl; 2-methylpropyl; iso-butyl; 1,1-dimethylethyl; tert-butyl; 1-methylbutyl; sec-pentyl; 2-methylbutyl; 3-methylbutyl; 1-ethylpropyl; 3-pentyl; 1,1-dimethylpropyl; tert-pentyl; 1,2-dimethylpropyl; 2,2-dimethylpropyl; neopentyl; 1-methylpentyl; 2-methylpentyl; 3-methylpentyl; 4-methylpentyl; iso-amyl; 1,1-dimethylbutyl; 1,2-dimethylbutyl; 1,3-dimethylbutyl; 2,2-dimethylbutyl; 2,3-dimethylbutyl, 3,3-dimethylbutyl; 1-ethylbutyl; 2-ethylbutyl; 1,1,2-trimethylpropyl; 1,2,2-trimethylpropyl; 1-ethyl-1-methylpropyl, 1-ethyl-2-methylpropyl; 1-methylhexyl; 2-methylhexyl; 3-methylhexyl; 4-methylhexyl; 5-methylhexyl; 1,1-dimethylpentyl; 1,2-dimethylpentyl; 1,3-dimethylpentyl; 1,4-dimethylpentyl; 2,2-dimethylpentyl; 2,3-dimethylpentyl; 2,4-dimethylpentyl; 3,3-dimethylpentyl; 3,4-dimethylpentyl; 4,4-dimethylpentyl; 1-ethylpentyl; 2-ethylpentyl; 3-ethylpentyl; 1,1,2-trimethylbutyl; 1,1,3-trimethylbutyl; 1,2,2-trimethylbutyl; 1,2,3-trimethylbutyl; 1,3,3-trimethylbutyl; 2,2,3-trimethylbutyl; 2,3,3-trimethylbutyl; 1-(methyl ethyl)butyl; 1-ethyl-1-methylbutyl; 1-ethyl-3-methylbutyl; 2-(methylethyl)butyl; 2-ethyl-1-methylbutyl; 2-ethyl-2-methylbutyl; 2-ethyl-3-methylbutyl; 1-propylbutyl; 2-propylbutyl; 1,1,2,2-tetramethylpropyl; 1-ethyl-1,2-dimethylpropyl; 1-ethyl-2,2-dimethylpropyl; 1-ethyl-1,2-dimethylpropyl; 1-methylheptyl; 2-methylheptyl; 3-methylheptyl; 4-methylheptyl; 5-methylheptyl; 6-methylheptyl; 1,1-dimethylhexyl; 1,2-dimethylhexyl; 1,3-dimethylhexyl; 1,4-dimethylhexyl; 1,5-dimethylhexyl; 2,2-dimethylhexyl; 2,3-dimethylhexyl; 2,4-dimethylhexyl; 2,5-dimethylhexyl; 3,3-dimethylhexyl; 3,4-dimethylhexyl; 3,5-dimethylhexyl; 4,4-dimethylhexyl; 4,5-dimethylhexyl; 5,5-dimethylhexyl; 1-ethylhexyl; 2-ethylhexyl; 3-ethylhexyl; 4-ethylhexyl; 1,1,2-trimethylpentyl; 1,1,3-trimethylpentyl; 1,1,4-trimethylpentyl; 1,2,2-trimethylpentyl; 1,2,3-trimethylpentyl; 1,2,4-trimethylpentyl; 1,3,3-trimethylpentyl; 1,3,4-trimethylpentyl; 1,4,4-trimethylpentyl; 2,2,3-trimethylpentyl; 2,2,4-trimethylpentyl; 2,3,3-trimethylpentyl; 2,3,4-trimethylpentyl; 2,4,4-trimethylpentyl; 3,3,4-trimethylpentyl; 3,4,4-trimethylpentyl; 1-ethyl-1-methylpentyl; 1-ethyl-2-methylpentyl; 1-ethyl-3-methylpentyl; 1-ethyl-4-methylpentyl; 2-ethyl-1-methylpentyl; 2-ethyl-2-methylpentyl; 2-ethyl-3-methylpentyl; 2-ethyl-4-methylpentyl; 3-ethyl-1-methylpentyl; 3-ethyl-2-methylpentyl; 3-ethyl-3-methylpentyl; 3-ethyl-4-methylpentyl; 1-propylpentyl; 2-propylpentyl; 1-(methylethyl)pentyl; 2-(methyl ethyl)pentyl; 3-(methyl ethyl)pentyl; 1,1,2,2-tetramethylbutyl; 1,1,2,3-tetramethylbutyl; 1,1,3,3-tetramethylbutyl; 1,2,2,3-tetramethylbutyl; 1,2,3,3-tetramethylbutyl; 2,2,3,3-tetramethylbutyl; 1-ethyl-1,2-dimethylbutyl; 1-ethyl-1,3-dimethylbutyl; 1-ethyl-2,2-dimethylbutyl; 1-ethyl-2,3-dimethylbutyl; 1-ethyl-3,3-dimethylbutyl; 2-ethyl-1,1-dimethylbutyl; 2-ethyl-1,2-dimethylbutyl; 2-ethyl-1,3-dimethylbutyl; 2-ethyl-2,3-dimethylbutyl; 2-ethyl-3,3-dimethylbutyl; 1,1-di ethylbutyl; 1,2-diethylbutyl; 2,2-di ethylbutyl; 1-methyl-1-propylbutyl; 2-methyl-1-propylbutyl; 3-methyl-1-propylbutyl; 1-methyl-1-(methylethyl)butyl; 2-methyl-1-(methyl ethyl)butyl; 3-methyl-1-(methyl ethyl)butyl; 1-methyl-2-(methylethyl)butyl; 2-methyl-2-(methyl ethyl)butyl; 3-methyl-2-(methyl ethyl)butyl; 1-(1,1-dimethylethyl)butyl; mixtures thereof; and like. Further examples of branched alkyl groups include 8-methylnonyl, and 3,5,5-trimethylhexyl.

Under one embodiment, the monomer is selected from the group consisting of hydroxypropyl acrylate, hydroxypropyl methacrylate, hydroxyethyl acrylate, hydroxyethyl methacrylate, ethyl acrylate, ethyl methacrylate, propyl acrylate, propyl methacrylate, butyl acrylate, butyl methacrylate, isobutyl acrylate, isobutyl methacrylate, sec-butyl acrylate, sec-butyl methacrylate, tetrahydrofurfuryl acrylate, tetrahydrofurfuryl methacrylate, caprolactone acrylate, caprolactone methacrylate, acroyloxyethyl maleate, methacroyloxyethyl maleate, and any mixture thereof.

The di(meth)acrylate as used in selected embodiments of the present invention is a diacrylate, a dimethacrylate, or a mixture thereof.

A diacrylate is a compound containing two acrylate group of formula CH₂═CH—CO—O—. A dimethacrylate is a compound containing two methacrylate group of formula CH₂═CMe—CO—O—.

Under one embodiment, the di(meth)acrylate of the present invention is selected from the group consisting of polyalkylene glycol diacrylate, aliphatic urethane diacrylate, aliphatic urethane dimethacrylate, alkoxylated bisphenol diacrylate, and alkoxylated bisphenol dimethacrylate.

Under one embodiment, polyalkylene glycol diacrylate is a compound of the formula CH₂═CH—CO—O—(CₙH₂ₙO)ₘ—(CO)—CH═CH₂, wherein m is 1 to 500, and n is 2 to 4. Examples of polyalkylene glycol diacrylate include polyethylene glycol diacrylate, polypropylene glycol diacrylate, and polybutylene glycol diacrylate.

Under one embodiment, polyethylene glycol diacrylate is a compound of formula CH₂═CH—CO—O—(C₂H₄O)ₘ—(CO)—CH═CH₂, wherein m is 1 to 500. Under one embodiment, 1<m<100. Under one embodiment, 1<m<50. Under one embodiment, 1<m<20. Under one embodiment, 1<m<10. Under one embodiment, 1<m<5. Under one embodiment, 5<m<10.

Under one embodiment, polypropylene glycol diacrylate is a compound of formula CH₂═CH—CO—O—(C₃H₆O)ₘ—(CO)—CH═CH₂, wherein m is 1 to 500. Under one embodiment, 1<m<100. Under one embodiment, 1<m<50. Under one embodiment, 1<m<20. Under one embodiment, 1<m<10. Under one embodiment, 1<m<5. Under one embodiment, 5<m<10. The —(C₃H₆O)— group may be any form known in the industry, such as —(CH₂—CH₂—CH₂—O)—, —(CH₂—CHMe—O)—, —(CHMe—CH₂—O)—, or any combination thereof.

Under one embodiment, polybutylene glycol diacrylate is a compound of formula CH₂═CH—CO—O—(C₄H₈O)ₘ—(CO)—CH═CH₂, wherein m is 1 to 500. Under one embodiment, 1<m<100. Under one embodiment, 1<m<50. Under one embodiment, 1<m<20. Under one embodiment, 1<m<10. Under one embodiment, 1<m<5. Under one embodiment, 5<m<10. The —(C₃H₆O)— group may be any form known in the industry, such as —(CH₂—CH₂—CH₂—CH₂—O)—, —(CH₂—CH₂—CHMe—O)—, —(CH₂—CHMe—CH₂—O)—, —(CHMe—CH₂—CH₂—O)—, —(CHMe—CHMe—O)—, —(CMe₂-CH₂—O)—, —(CH₂—CMe₂-O)—, or any combination thereof.

Aliphatic urethane di(meth)acrylate is a hydrocarbyl compound comprising two (meth)acrylate groups and at least one urethane group. Urethane group is —NH—CO—O— bridging group. A generic formula for aliphatic urethane di(meth)acrylate, under one embodiment, is CH₂═CH—CO—O—[(CRR′)ᵢ—(NH—CO—O)ⱼ—(O—CO—NH)ₖ]_{L}—(CO)—CH═CH₂, wherein i+j+k=1, L=1 to 100.

Alkoxylated bisphenol (meth)acrylate is a compound comprising two (meth)acrylate groups, at least one bisphenol group, and —(CₙH₂ₙO)— groups wherein n is 2 to 4.

Examples of alkoxylated bisphenol (meth)acrylate include ethoxylated (r) bisphenol A diacrylate, wherein (r) is the number of ethoxylene groups. For example, ethoxylated (3) bisphenol A diacrylate has the formula

CH₂═CH—CO—O—(C₂H₄O)ₛ—C₆H₄—CMe₂- C₆H₄—(OC₂H₄)ₜ—O—(CO)—CH═CH₂

wherein s+t=3.

Under one embodiment, the dental coating composition comprises a (meth)acrylate monomer, a (meth)acrylate dimer, a (meth)acrylate crosslinker, a photoinitiator, and a silicone surfactant.

Under one embodiment the dental coating comprises a crosslinker. Under another embodiment the dental coating comprises two or more different compounds that are crosslinkers.

Under one embodiment, the crosslinker is aliphatic urethane poly(meth)acrylate or ethoxylated (meth)acrylate.

The crosslinker has a functionality of greater than about 2.5. Under one embodiment, the functionality of the crosslinker is equal to or greater than 3.0 and less than 5.0. Under one embodiment, the functionality of the crosslinker is equal to or greater than 4.0 and less than 6.0. Under one embodiment, the functionality of the crosslinker is equal to or greater than 5.0 and less than 9.0. Under one embodiment, the functionality of the crosslinker is equal to or greater than 8.0 and less than 12.0. Under one embodiment, the functionality of the crosslinker is equal to or greater than 10.0 and less than 15.0.

Generally, a crosslinker is a multifunctional (meth)acrylate. Examples of crosslinkers include tri(meth)acrylates, tetra(meth)acrylates, penta(meth)acrylates and higher (meth)acrylates. Such examples include trimethylolpropane triacrylate, trimethylolethane triacrylate, trimethylolpropane trimethacrylate, trimethylolethane trimethacrylate, tetramethylene glycol dimethacrylate, triethylene glycol dimethacrylate, tetraethylene glycol diacrylate, pentaerythritol diacrylate, penta-erythritol triacrylate, pentaerythritol tetraacrylate, dipentaerythritol diacrylate, dipenta-erythritol triacrylate, dipentaerythritol tetraacrylate, dipentaerythritol pentaacrylate, dipenta-erythritol hexaacrylate, tripentaerythritol octaacrylate, pentaerythritol dimethacrylate, penta-erythritol trimethacrylate, dipentaerythritol dimethacrylate, dipentaerythritol tetramethacrylate, tripentaerythritol octamethacrylate, ethylene glycol diacrylate, ethylene glycol dimethacrylate, 1,3-butanediol diacrylate, 1,3-butanediol dimethacrylate, sorbitol triacrylate, sorbitol tetraacrylate, pentaerythritol-modified triacrylate, sorbitol tetramethacrylate, sorbitol pentaacrylate, sorbitol hexaacrylate, oligoester acrylates and methacrylates, glycerol di- and tri-acrylate, 1,4-cyclohexane diacrylate, bisacrylates and bismethacrylates of polyethylene glycol, and mixtures thereof.

Under one embodiment, at least one of the one or more crosslinker is selected from the group consisting of trimethylol propane tri(meth)acrylate, ethoxylated glycerin tri(meth)acrylate, tricyclodecane dimethanol diacrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated isocyanuric acid tri(meth)acrylates.

Under one embodiment the crosslinkers comprise methacrylate groups. Example of such crosslinkers include dimethacrylates, trimethacrylates, tetramethacrylate, and higher methacrylates. Examples of such methacrylic crosslinkers include trimethylolpropane trimethacrylate, trimethylolethane trimethacrylate, tetramethylene glycol dimethacrylate, triethylene glycol dimethacrylate, pentaerythritol dimethacrylate, penta-erythritol trimethacrylate, dipentaerythritol dimethacrylate, dipentaerythritol tetramethacrylate, tripentaerythritol octamethacrylate, 1,3-butanediol dimethacrylate, sorbitol tetramethacrylate, oligoester methacrylates, bismethacrylates of polyethylene glycol having a molecular weight of from 200 to 1500, and mixtures thereof. For example, trimethylolpropane trimethacrylate is a composition consisting of, or comprising largely of, the compound of formula (CH₂═CMe—C(O)—O—CH₂)₃C—C₂H₅. It is a low volatility trifunctional monomer offering fast cure response in free radical polymerization.

Another suitable crosslinker is an alkoxylated crosslinker, with the formula (CH₂═CMe—C(O)—O—(AO)ₓCH₂—)₃C—R; wherein wherein R is a C₁ to C₆ alkyl group; AO is a small alkoxy group, such as an ethylene oxide, —CH₂—CH₂—O—, propylene oxide, —CH(CH₃)—CH₂—O—, —CH₂—CH₂—CH₂—O—, butylene oxide, and —CH(Et)—CH₂—O—; and wherein for each (CH₂═CMe—C(O)—O—(AO)ₓ—CH₂—) group x is independently 0, 1, 2, or 3. Using R=ethyl, and AO=ethylene oxide as an example, an exemplary alkoxylated crosslinker has a structure of formula

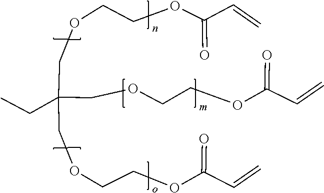

wherein m, n, and o are each independently 0, 1, 2, or 3.

The photopolymerizable composition of the present invention for forming a dental coating for dental prosthetics also optionally comprises one or more photoinitiators. As described below, the photoinitiator is selected so that it is activated by photons of the wavelength associated with actinic light of a UV lamp, an LED lamp, or another lamp. Preferably, the photoinitiator should be active at the wavelength of actinic light of a UV lamp, an LED lamp or another lamp that also cures the partially cured dental prosthetics.

Such photoinitiators may be selected from benzyl ketones, monomeric hydroxyl ketones, polymeric hydroxyl ketones, α-amino ketones, acyl phosphine oxides, metallocenes, benzophenone, and benzophenone derivatives. Specific examples of photoinitiators include 1-hydroxy-cyclohexylphenylketone; benzophenone; 2-benzyl-2-(dimethylamino)-1-(4-(4-morphorlinyl)phenyl)-1-butanone; 2,2-dimethoxy-2-phenyl acetophenone; 2-methyl-1-(4-methylthio)phenyl-2-(4-morphorlinyl)-1-propanone; 2,4,6-trimethylbenzoyldiphenyl-phosphine oxide; bis(2,4,6-trimethyl benzoyl)phenyl phosphine oxide; diphenyl-(2,4,6-trimethylbenzoyl) phosphine oxide; bis(2,6-dimethoxybenzoyl-2,4,4-trimethyl pentyl)phosphine oxide; 2-hydroxy-2-methyl-1-phenyl-propan-1-one; phenyl bis(2,4,6-trimethylbenzoyl) phosphine oxide; benzyl-dimethylketal; isopropylthioxanthone; bis(η⁵-2,4-cyclopentadien-1-yl)bis[2,6-difluoro-3-(1H-pyrrol-1-yl)phenyl]titanium), and mixtures of any of the foregoing. Under one embodiment of the present invention, the photopolymerizable composition comprises a single chemical compound that exhibits photoinitiating properties. Under an alternative embodiment, the photoinitiator is a mixture of photoinitiators.

The photoinitiator is present in the photopolymerizable composition in amounts sufficient to be effective in aiding curing of the photopolymerizable composition. Such amounts may be determined empirically. The photopolymerizable composition comprises up to about 10 wt % of one or more photoinitiators. Under one embodiment, the photopolymerizable composition comprises about 0.5 to about 5.0 wt % of one or more photoinitiators.

The photopolymerizable composition of the present invention under one embodiment further comprises a small amount of a colorant or special effects pigment or a combination thereof.

One purpose of using pigment in the photopolymerizable composition is to provide a tint or a color to the dental coating.

Another purpose of using a pigment is to give a clear or colorless or whitish appearance of the dental coating.

Examples of pigments may be incorporated into the photopolymerizable composition of the present invention include: annatto, caramel, carmine, β-carotene, potassium sodium copper chlorophyllin (chlorophyllin copper-complex), dihydroxyacetone, bismuth oxychloride, guaiazulene, iron oxides, ferric ammonium ferrocyanide, ferric ferrocyanide, chromium hydroxide green, chromium oxide greens, guanine, pyrophyllite, mica, silver, titanium dioxide, aluminum powder, bronze powder, copper powder, ultramarines, manganese violet, zinc oxide, luminescent zinc sulfide, FD&C Blue No. 1, D&C Blue No. 4, Iron Blue, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, and mixture of any of the preceding. As will be recognized by the practitioner of the art, some of the pigments in the above list are better suited for use in the photopolymerizable composition than others, because they offer better composition stability of the photopolymerizable composition, and they do not interfere with the UV curing process.

Under one embodiment the photopolymerizable composition comprises the pigment is selected from the group consisting of ultramarine, manganese violet, zinc oxide, FD&C Blue No. 1, D&C Blue No. 4, Iron Blue, D&C Violet No. 2, and a mixture thereof.

Special effects pigment may be any pigment that gives either the photopolymerizable composition or the formed cured composition a special effect, such as an increased pearlescent, iridescent, shimmering, transparency or complex effects. Examples of special effect pigments include titanated micas, mica-based interference colors, mica coated with titanium dioxide and iron oxide, mica-based gold pearls, mica-based metallic pearls, mica-based pearl pigments, bismuth oxychloride, synthetic mica-based interference pearls, synthetic mica-based white pigment, silicate-based pearls, titanium oxide and tin oxide on silicate platelets, flaked aluminum powder, silver coated silicate flakes, and any combination of the foregoing.

The photopolymerizable composition of the present application may further comprise additional ingredients, including colorants, dyes, whiteners, perfumes, thixotropes, stabilizers, anti-oxidants, and like.

The dental coating composition of the present invention for forming a dental coating for dental prosthetics also comprises one or more silicone surfactants.

Silicone surfactants are selected to reduce the surface tension of the dental coating composition. A silicone surfactant is a polysiloxane, a polymers comprising siloxane groups, or formula (R₂Si—O—SiR₂). Examples of silicone surfactants include polydialkylsiloxanes, polydimethylsiloxanes, polydiethylsiloxanes, polydipropylsiloxanes, and like. Further, silicone surfactants also include polyalkylene-modified polydialkylsiloxanes,

The photopolymerizable composition of the present invention for forming a dental coating for dental prosthetics optionally comprises a filler or a thickener.

The filler is used to adjust the viscosity of the dental coating. The viscosity will, in turn, determine the thickness of the resulting dental coating. Examples of fillers include silica, zirconia, alumina, calcium carbonate, calcium phosphates, and mixtures thereof.

Under one embodiment, the dental coating comprises particles. These particles are selected from the group consisting of fibers, flakes, sheets, crystals, and rods.

One purpose of incorporating particles in the dental coating formulation is to provide a “character” to the surface of the dental prosthetic. One purpose of incorporating particles in the dental coating formulation is to provide an esthetic appeal of the dental prosthetic. The dental coating thus may make the parts of the dental prosthetic appear to approximate gingivae or gum.

Such particles may be selected to approximate blood capillaries, or to model blood capillaries. Under one embodiment, the particle is a fine fiber. Under one embodiment, a fiber is 1 to 5 mm long, or 1.5 to 3.0 mm long. The fibers are 5 μm to 20 μm in thickness, or 10 μm to 15 μm in thickness.

The color of the fibers are selected to approximate the color of capillaries. Suitable colors include red, cardinal, coral, crimson, maroon, rose, wine, blush, brick, burgundy, carmine, cerise, cherry, claret, copper, garnet, geranium, magenta, puce, ruby, russet, rust, salmon, sanguine, scarlet, titian, vermilion, bloodshot, florid; blue, azure, beryl, cerulean, cobalt, indigo, navy, royal, sapphire, turquoise, and ultramarine.

The fibers may be composed of any material that is suitable for use in dental prosthetics. Examples of such fibers include nylon, PVC, acetate, acrylic, olefin, polyester, rayon, glass, metal, and a mixture thereof.

The fibers also are of sufficient flexibility to allow for encapsulation within the coating. The fibers under one embodiment do not stick out of the dental coating.

The coating composition, under one embodiment, comprises a variety of fibers. The fibers vary with color, length, thickness, and other characteristics, to yield a coating exhibiting heterogeneity of capillary models.

The present invention is also directed to a dental prosthesis which comprises the dental coating.

The phrase “dental prosthesis”, refers to an intraoral prosthesis. Such a prosthesis may be used to restore intraoral defects such as missing teeth, missing parts of teeth, and missing soft or hard structures of the jaw and palate. A dental prosthesis may be used to rehabilitate mastication, improve aesthetics, and aid speech. A dental prosthesis may be held in the mouth by connecting to teeth or to dental implants, by suction, or it may be held passively by surrounding muscles.

Under one embodiment, the dental prosthesis is a removable dental prosthesis. Under one embodiment, the dental prosthesis is a fixed dental prosthesis.

A dental prosthesis may be either be fixed permanently or it may be removable. Permanently fixed dental prostheses use dental adhesive or screws, to attach to teeth or to dental implants. Removal prostheses may use friction against parallel hard surfaces and undercuts of adjacent teeth or dental implants, suction using the mucous retention (with or without aid from denture adhesives), and by exploiting the surrounding muscles and anatomical contours of the jaw to passively hold in place.

As is understood from the context, the term “dental prosthesis” may refer to a dental prosthesis that has no coating on it, or a dental prosthesis that already one or more layers of a dental coating composition on it, or a dental prosthesis that comprises a dental coating.

The dental prosthesis comprising the dental coating is a denture, a partial denture, a palatal obturator, an orthodontic appliance, a dental implant, a crown, or a bridge. Under one embodiment, the dental prosthesis is a denture, a partial denture, a palatal obturator, or a dental implant.

A denture is dental prosthetic device constructed to replace missing teeth and is supported by the surrounding soft and hard tissues of the oral cavity. Conventional dentures are removable. Such dentures may be removable. A denture may be a partial denture or a complete denture. The examples of a denture also include a maxillary denture and a mandibular denture.

The term “denture” also includes any form of a denture, such as a final denture, a provisional denture, an immediate denture, a trial denture, or a verification trial denture. A trial denture is the arrangement of teeth in wax, for trial, prior to the completion of the denture.

Under one embodiment, the dental prosthesis is used in the treatment of patients that are either partially edentulous or fully edentulous.

The present invention is also directed to a method of forming a dental prosthesis.

One advantage of the present invention over prior art is the speed and ease of finishing the unfinished dental prosthesis. To finish manufacturing of the dental prosthesis, whether 3D printed or manufactured in another way, such a prosthesis needed to be polished to have a glossy surface appearance and optionally “characterized” with acrylic stains to give the prosthesis more a more esthetically pleasing look. To do the finishing work, the manual labor time was about 5 to 15 minutes per arch, plus the finishing materials that are consumed.

The present inventive method allows the dental prosthetics to be finished with the dental coating, rather than the polishing described above. Finishing of the prosthesis takes a fraction of the time and material consumption to get to a similar or better esthetic result.

The use of the dental coating composition is so easy, fast, and cheap that this invention is suitable not only for a final dental prosthetic but also for a trial dental prosthetic. For example, a trial denture is used to check how the dental prosthetic looks, feels, and functions in the patient before the final denture is fabricated. The trial prosthetic is a monolithic 3D printed device that is tooth-colored material. To get a realistic look, and before being placed in the patient, the gum line is “characterized” to add pink/red/brown shading as needed to give the gums a natural appearance for the trial process. The process of this invention does this better, quicker, and cheaper than the prior art processes.

The present invention is also directed to several methods of forming a dental prosthesis. Under the Coated Acrylic Denture Method, the method includes at least three steps: (a) obtaining an unfinished dental prosthesis, wherein the unfinished dental prosthesis comprises a surface of the unfinished dental prosthesis; (b) applying the dental coating composition onto a part of the surface; and (c) curing the coating.

The phrase “unfinished dental prosthesis” refers to a dental prosthesis that is manufactured through any of various methods, prior to the application of the coating composition of the present invention.

The definition of “unfinished dental prosthesis” also includes any dental prosthesis which has not been yet coated with the dental coating composition of the present invention. Such a dental prosthesis may be in the state that is acceptable to the patient.

The unfinished dental prosthesis may be obtained by any suitable method. Such a method includes currently known methods, or methods that may be developed in the future that produce unfinished dental prosthesis.

Examples of methods used to obtain the unfinished dental prosthesis include fabrication by additive manufacturing, 3D printing, subtractive manufacturing, milling, self-cured fabrication process, heat-cured fabrication process, or injection molding.

The process may include a traditional method of forming prosthetics such as dentures. Under such a method, a dentist makes an impression of the patient's oral cavity. A plaster model of the oral cavity is made using that impression. The model is placed on an articulator, and a technician attaches chosen teeth with wax. The technician shapes and carves the wax to represent parts of the oral cavity, including the teeth and gum. After adjusting this model denture with input by the dentist and the patient, the model dentures are then placed in a flask holding device. Plaster is poured in to maintain the shape of the dentures, and the flask is then placed in boiling water to melt away the wax. Any leftover wax is rinsed away. The technician paints a liquid separator onto the plaster, so the acrylic doesn't stick to it. Acrylic is then injected into the flask to replace the wax and is cured. The technician carefully breaks off the plaster mold. The dentures are then placed into an ultrasonic bath to ensure all remnants of plaster are removed. The technician trims any excess acrylic from the denture and then polishes it with pumice. The dentures are finished and are sent to the dentist for the patient's initial fitting.

Under an alternative embodiment, the unfinished dental prosthesis is obtained by an additive manufacturing technique. An additive manufacturing technique under one embodiment is a 3D printing process. Examples of additive manufacturing techniques include a vat polymerizable technique, a fusion deposition technique, a material jetting technique, a nanoparticle jetting technique, and a drop-on-demand technique.

Under one embodiment, examples of suitable additive manufacturing techniques include stereolithography (SLA), digital light processing (DLP), and continuous digital light processing (CDLP).

Under one embodiment, the additive manufacturing technique is digital light processing.

Under one embodiment, the additive manufacturing technique comprises the use of a liquid photopolymer resin.

The unfinished dental prosthesis comprises a surface. The surface includes any surface between the dental prosthesis and the surrounding medium, such as air, liquid, and oral cavity.

After the unfinished dental prosthesis is obtained, the dental coating composition is applied. The application is performed by painting the part of the surface of the unfinished dental prosthesis by any suitable applicator. Examples of suitable applicators include a brush and a sponge. Under one embodiment, the brush is similar to the type of brush typically used to apply nail polish.

Under one embodiment, the part of the surface to which dental coating composition is applied to is the portion of the surface that mimics the gingivae.

After the application of the dental coating composition to a part of the surface of the dental prosthesis, the dental coating composition is cured. The curing process may be done by any of suitable processes, including exposure to heat, exposure to sunlight, exposure to UV light lamps, exposure to LED light lamps, exposure to actinic light.

The process of forming the dental prosthesis wherein the unfinished dental prosthesis is formed by additive manufacture under certain embodiments includes additional steps. Such steps include any of the following steps: (1) washing the residual liquid photopolymer resin from the unfinished dental prosthesis, (2) polishing the unfinished dental prosthesis, or (3) sandblasting the unfinished cured dental prosthesis. Any of these three steps (1), (2), and (3) may be performed. The performance may be executed in any order, before, after, or in-between any of the steps (a), (b), or (c).

The present invention is also directed to a Coated/Post-Cured method of forming a dental prosthesis that includes at least the following four steps: (a) obtaining a partially-cured dental prosthesis, wherein the partially-cured denture comprises a surface; (b) curing the partially-cured dental prosthesis; (c) applying the dental coating composition onto a part of the surface; and (d) curing the dental coating composition.

One advantage of using this method is that the curing step (b) and (d) may be performed by different techniques under different conditions.

The phrase “partially-cured dental prosthesis” refers to a dental prosthesis that is manufactured through additive manufacturing technique. The “partially-cured” designation is also colloquially known in the art as a “green-state”.

Under one embodiment, the partially-cured dental prosthesis is about 60% to about 95% cured. Under one embodiment, the partially-cured dental prosthesis is about 70% to about 93% cured. Under one embodiment, the partially-cured dental prosthesis is about 80% to about 89% cured.

The methods used to obtain the partially-cured dental prosthesis is fabrication by additive manufacturing. An additive manufacturing technique under one embodiment is a 3D printing process. Examples of additive manufacturing techniques include a vat polymerizable technique, a fusion deposition technique, a material jetting technique, a nanoparticle jetting technique, and a drop-on-demand technique.

Under one embodiment, examples of suitable additive manufacturing techniques include stereolithography (SLA), digital light processing (DLP), and continuous digital light processing (CDLP). Under one embodiment, the additive manufacturing technique is digital light processing. Under one embodiment, the additive manufacturing technique comprises the use of a liquid photopolymer resin.

Under one embodiment, the additive manufacturing technique is digital light processing. Under one embodiment, the additive manufacturing technique comprises the use of a liquid photopolymer resin. The partially-cured dental prosthesis comprises a surface. The surface includes any surface between the dental prosthesis and the surrounding medium, such as air, liquid, and oral cavity.

After the partially-cured dental prosthesis is obtained, but before the dental coating composition is applied, the partially-dental prosthesis is cured. The curing process may be done by any of suitable processes, including exposure to heat, exposure to sunlight, exposure to UV light lamps, exposure to LED light lamps, exposure to actinic light, or any combination of the these.

After the partially-cured dental prosthesis is cured, the dental coating composition is applied thereto. The application is performed by painting the part of the surface of the unfinished dental prosthesis with any suitable applicator. Examples of suitable applicators include a brush and a sponge. Under one embodiment, the brush is similar to the type of brushed typically used to apply nail polish. Under one embodiment, the part of the surface to which dental coating composition is applied to is the portion of the surface that mimics the gingivae.

After applying the dental coating composition to a part of the surface of the dental prosthesis, the dental coating composition is cured. The curing process may be done by any suitable processes, including exposure to heat, exposure to sunlight, exposure to UV light lamps, exposure to LED light lamps, exposure to actinic light.

The process of forming the dental prosthesis wherein the partially-cured dental prosthesis is formed by additive manufacture under certain embodiments includes additional steps. Such steps include any of the following steps: (1) washing the residual liquid photopolymer resin from the partially-cured dental prosthesis, (2) polishing the partially-cured dental prosthesis, or (3) sandblasting the partially-cured dental prosthesis. Any of these three steps (1), (2), and (3) may be performed. The performance may be executed in any order, before, after, or in-between any of the steps (a), (b), (c) or (d).

The present invention is also directed to a Green State Coated method of forming a dental prosthesis that includes at least the following three steps: (a) obtaining a partially-cured dental prosthesis, wherein the partially-cured denture comprises a surface; (b) applying the dental coating composition of any of the claims 1 to 200 onto a part of the surface; and (c) curing the partially-cured denture and the dental coating composition.

One advantage of using this method is that the curing step (c) cures both the partially-cured denture and the dental coating composition at the same time. This combination lowers the time to cure the partially-cured dental prosthesis. Further, this combination has been found to provide superior adhesion properties.

To improve the curing steps process, under one embodiment, the curing of the partially-cured dental prosthesis is well-matched with the curing of the dental coating composition.

Under one embodiment, the partially-cured dental prosthesis is about 60% to about 95% cured. Under one embodiment, the partially-cured dental prosthesis is about 70% to about 93% cured. Under one embodiment, the partially-cured dental prosthesis is about 80% to about 89% cured. Under one embodiment, the partially-cured dental prosthesis is about 60% to about 70% cured. Under one embodiment, the partially-cured dental prosthesis is about 70% to about 75% cured. Under one embodiment, the partially-cured dental prosthesis is about 75% to about 80% cured. Under one embodiment, the partially-cured dental prosthesis is about 80% to about 83% cured. Under one embodiment, the partially-cured dental prosthesis is about 83% to about 87% cured. Under one embodiment, the partially-cured dental prosthesis is about 87% to about 93% cured.

The methods used to obtain the partially-cured dental prosthesis is a fabrication by additive manufacturing. An additive manufacturing technique under one embodiment is a 3D printing process. Examples of additive manufacturing techniques include a vat polymerizable technique, a fusion deposition technique, a material jetting technique, a nanoparticle jetting technique, and a drop-on-demand technique.

Under one embodiment, examples of suitable additive manufacturing techniques include stereolithography (SLA), digital light processing (DLP), and continuous digital light processing (CDLP). Under one embodiment, the additive manufacturing technique is digital light processing. Under one embodiment, the additive manufacturing technique comprises the use of a liquid photopolymer resin.

Under one embodiment, the additive manufacturing technique is digital light processing. Under one embodiment, the additive manufacturing technique comprises the use of a liquid photopolymer resin. The partially-cured dental prosthesis comprises a surface. The surface includes any surface between the dental prosthesis and the surrounding medium, such as air, liquid, and oral cavity.

The dental coating composition is applied to the partially-cured dental prosthesis. The application is performed by painting the part of the surface of the unfinished dental prosthesis by any suitable applicator. Examples of suitable applicators include a brush and a sponge. Under one embodiment, the brush is similar to the type of brush typically used to apply nail polish. Under one embodiment, the part of the surface to which dental coating composition is applied to is the portion of the surface that mimics the gingivae.

After applying the dental coating composition to a part of the surface of the dental prosthesis, the dental coating composition and the partially-cured dental prosthesis are cured concurrently. The curing process may be done by any of suitable processes, including exposure to heat, exposure to sunlight, exposure to UV light lamps, exposure to LED light lamps, exposure to actinic light.

Under one embodiment, the curing of the partially-cured dental prosthesis and the dental coating composition proceeds concurrently.

Under another embodiment, the curing process in 2 or more sub-steps, each which may be optimized for their level of curing. This may occur in cases wherein the curing process that is viable for the curing of the partially-cured dental prosthesis does not appreciably cure the dental coating, or in cases wherein the curing process that is viable for the curing of the dental coating prosthesis does not appreciably cure the partially-cured dental prosthesis.

The process of forming the dental prosthesis wherein the partially-cured dental prosthesis is formed by additive manufacture under certain embodiments includes additional steps. Such steps include any of the following steps: (1) washing the residual liquid photopolymer resin from the partially-cured dental prosthesis, (2) polishing the partially-cured dental prosthesis, or (3) sandblasting the partially-cured dental prosthesis. Any of these three steps (1), (2), and (3) may be performed. The performance may be executed in any order, before, after, or in-between any of the steps (a), (b), or (c).

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. A dental coating composition comprising:
(a) about 5 wt % to about 20 wt % of a (meth)acrylate monomer selected from the group consisting of alkyl (meth)acrylate and heteroalkyl (meth)acrylate;
(b) about 30 w % to about 65 wt % of a di(meth)acrylate selected from the group consisting of polyalkylene glycol diacrylate, aliphatic urethane di(meth)acrylate, and alkoxylated bisphenol (meth)acrylate;
(c) about 25 wt % to about 40 wt % of (meth)acrylate crosslinker selected from the group consisting of aliphatic urethane poly(meth)acrylate and ethoxylated (meth)acrylate, wherein the (meth)acrylate crosslinker has a functionality greater than about 2.5;
(d) a photoinitiator; and
(e) a silicone surfactant.

2. (canceled)

3. The dental coating of claim 1, wherein the monomer is selected from the group consisting of hydroxypropyl acrylate, hydroxypropyl methacrylate, hydroxyethyl acrylate, hydroxyethyl methacrylate, ethyl acrylate, ethyl methacrylate, propyl acrylate, propyl methacrylate, butyl acrylate, butyl methacrylate, isobutyl acrylate, isobutyl methacrylate, sec-butyl acrylate, sec-butyl methacrylate, tetrahydrofurfuryl acrylate, tetrahydrofurfuryl methacrylate, caprolactone acrylate, caprolactone methacrylate, acroyloxyethyl maleate, methacroyloxyethyl maleate, and any mixture thereof.

4. The dental coating of claim 1, further comprising a filler selected from the group consisting of silica, zirconia, alumina, calcium carbonate, calcium phosphates, and mixtures thereof.

5-6. (canceled)

7. The dental coating of claim 1, further comprising particles selected from the group consisting of fibers, flakes, sheets, crystals and rods.

8. The dental coating of claim 1, wherein the particles are fibers, wherein the fibers are selected from the group consisting of nylon, PVC, acetate, acrylic, olefin, polyester, rayon, glass, metal, and a mixture thereof.

9. A dental coating composition comprising:
(a) about 52 w % to about 75 wt % of a di(meth)acrylate selected from the group consisting of polyalkylene glycol diacrylate, aliphatic urethane di(meth)acrylate, and alkoxylated bisphenol (meth)acrylate;
(b) about 20 wt % to about 43 wt % of (meth)acrylate crosslinker selected from the group consisting of aliphatic urethane poly(meth)acrylate and ethoxylated (meth)acrylate, wherein the (meth)acrylate crosslinker has a functionality greater than about 2.5;
(c) a photoinitiator; and
(d) a silicone surfactant.

10-13. (canceled)

14. The dental coating of claim 9, further comprising a colorant.

15. The dental coating of claim 9, further comprising a brightener.

16. The dental coating of claim 9, further comprising particles selected from the group consisting of fibers, flakes, sheets, crystals, and rods.

17. The dental coating of claim 9, wherein the particles are fibers, wherein the fibers are selected from the group consisting of nylon, PVC, acetate, acrylic, olefin, polyester, rayon, glass, metal, and a mixture thereof.

18. A dental prosthesis comprising a dental coating of claim 1.

19. The dental prosthesis of claim 18, wherein the dental prosthesis is a denture, a partial denture, a palatal obturator, an orthodontic appliance, a dental implant, a crown, or a bridge.

20-24. (canceled)

25. A method of forming a dental prosthesis, including the steps of
(a) obtaining an unfinished dental prosthesis, wherein the unfinished dental prosthesis comprises a surface;
(b) applying the dental coating composition of any of the claims 1 to 17 onto a part of the surface; and
(c) curing the dental coating composition.

26. The method of claim 25, wherein the curing of the coating comprises exposure to actinic light.

27. The method of claim 25, wherein the unfinished dental prosthesis is fabricated by additive manufacturing, 3D printing, subtractive manufacturing, milling, self-cured fabrication process, heat-cured fabrication process, or injection molding.

28. The method of claim 25, wherein the unfinished dental prosthesis is obtained by an additive manufacturing technique.

29. The method of claim 28, wherein the additive manufacturing technique is selected from the group consisting of a vat polymerizable technique, a fusion deposition technique, a material jetting technique, a nanoparticle jetting technique, and a drop-on-demand technique.

30. The method of claim 28, wherein the additive manufacturing technique is selected from the group consisting of stereolithography, SLA, digital light processing, DLP, continuous digital light processing, and CDLP.

31-32. (canceled)

33. The process of claim 25, wherein the procedure further comprises any of the following steps: washing the residual liquid photopolymer resin from the partially-cured dental prosthesis, polishing the partially-cured dental prosthesis, and sandblasting the partially-cured dental prosthesis.

34-53. (canceled)

54. A dental prosthesis comprising a dental coating of claim 9.
